(19) United States
(12) Patent Application Publication
(10) Pub. No.: US 2021/0322228 A1
(43) Pub. Date: Oct. 21, 2021

(54) Sealskin Application System

(71) Applicants: Annamarie Joseph, Charlotte, NC (US); Lawrence Bergenfield, Las Vegas, NV (US); Theodore Search, Conestoga, PA (US)

(72) Inventors: Annamarie Joseph, Charlotte, NC (US); Lawrence Bergenfield, Las Vegas, NV (US); Theodore Search, Conestoga, PA (US)

(21) Appl. No.: 16/848,988

(22) Filed: Apr. 15, 2020

(51) Int. Cl. A61F 13/00

(52) U.S. Cl. CPC A61F 13/00072; A61F 13/00063; A61F 13/00068

(57) ABSTRACT

A system for providing a protective covering for a human or non-human animal comprising a supply of dispensable plastic stretch film pre-wound onto a spool and a dispenser-applicator for dispensing the film and applying the film to the human or non-human animal, wherein the surface of the side of film applied to the human or non-human animal is coated with a mixture of manuka honey, activated charcoal, and tea tree oil.

### BACKGROUND OF THE INVENTION

State-of-the-art medical care requires that open wounds, plaster and other types of casts, porous and non-waterproof bandages and the like be protected by watertight coverings that also prevent access to the injured areas by infectious agents.

### BRIEF SUMMARY OF THE INVENTION

The present invention relates to a system and method for providing and applying a waterproof, moisture-sealing protective film cover to a portion of the anatomy of a human or non-human animal in need thereof, such as, for example, one bearing a wound, cast, prosthesis, dressing, bandage, compress, drainage tube, “covering & waterproofing IV PICC line area, or the like.

One embodiment of the invention relates to a system and method for providing a protective covering for at least a portion of a human or non-human animal comprising a supply of dispensable plastic stretch film having a predetermined thickness and width and a dispenser-applicator for dispensing a predetermined length of film from the supply thereof and applying the predetermined length of film to the human or non-human animal, wherein the surface of the side of film adapted for application to the human or non-human animal is coated with a wound healing composition, preferably, a mixture of manuka honey, activated charcoal, and tea tree oil.

An additional embodiment of the invention may be the above described system and method wherein the dispenser-applicator is coupled to and longitudinally aligned with a telescoping mechanism which enables extension of the supply of dispensable plastic stretch film.

A further embodiment of the invention may be an article of manufacture comprising packaging material and at lest one of the above-described systems for providing a protective covering for at least a portion of a human or non-human animal contained within the packaging material, wherein the packaging material comprises a label indicating that the system(s) can be used for providing a protective covering for at least a portion of a human on non-human animal and instructions for providing the protective covering employing the system(s)..

Additional embodiments of the invention may relate to wound-healing compositions comprising a mixture of manuka honey, activated charcoal, and tea tree oil and methods for use thereof to enhance wound healing.

### BRIEF DESCRIPTION OF THE DRAWINGS

Other objects, features, and advantages of the present invention will be apparent from the written description and the drawings in which:

FIG. 1 shows a perspective view of dispenser-applicator systems in accordance with an embodiment of the invention;

FIG. 1A shows a side view of dispenser-applicators depicted in FIG. 1.

FIG. 2 is an enlarged exploded isometric view showing the various parts of a telescoping mechanism in disassembled condition;

FIG. 3 is an enlarged fragmentary longitudinal section through the telescoping mechanism of FIG. 2.

### DETAILED DESCRIPTION OF THE INVENTION

It is known that each of manuka honey, activated charcoal, and tea tree oil are useful as wound healing agents.

The present invention is predicated on the unexpected discovery that a mixture of manuka honey, activated charcoal and tea tree oil possesses wound healing properties synergistically superior to any of the individual ingredients, either alone or in mixtures of any two thereof.

More particularly, the systems and methods described above which enable the application of a waterproof, moisture-sealing protective film cover to a portion of the anatomy of a human or non-human animal in need thereof, such as, for example, one bearing a wound, cast, prosthesis, dressing, bandage, compress, drainage tube, or the like, are greatly improved by the present invention, which relates to a coating on the surface of the side of film applied to the human or non-human animal with a mixture of manuka honey, activated charcoal, and tea tree oil.

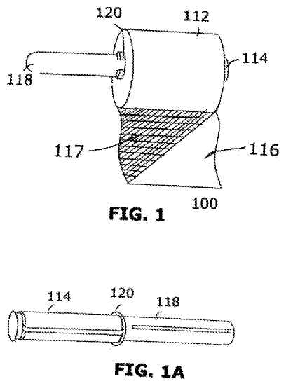

Referring to FIG. 1, there is depicted dispenser-applicator system 100 which consists of a supply of plastic stretch film 112 preferably pre-wound onto spool 114, which is mounted on preferably ergonomic handle 118, which may be of any convenient length to accommodate dispensing of the film 112. The interior side 116 of the film 112 is coated with the above described mixture of manuka honey, activated charcoal, and tea tree oil, shown partially as cross-hatched area 117 for illustration purposes, it being understood by those skilled in the art that coating 117 may extend over the entire surface of interior side 116 or any desired fraction or part thereof.

As depicted in FIG. 1A, in dispenser-applicator 100 handle 118 may be of any desired length and may, optionally, slidingly and longitudinally extend within spool 114 and coupled thereto with telescoping mechanism 120 which allows, if desired, extending the spool 114 to enable application of the film 112 to hard-to-reach areas.

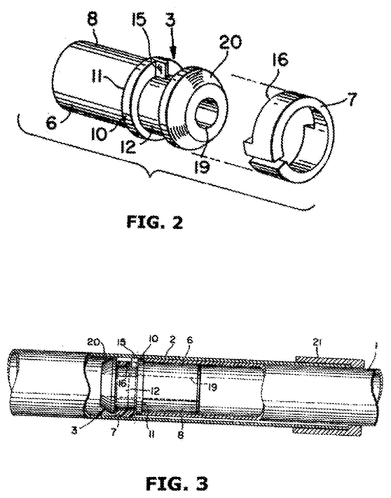

In FIGS. 2 and 3 there is shown a preferred telescoping mechanism comprising a pair of telescoping members 1 and 2 (corresponding to elements 114 and 118 in FIGS. 1 and 1A), which may be releasably locked in longitudinal adjusted position using cam lock 3. It will be understood by those skilled in the art that locking the telescoped device in any position will not affect the sealskin applicating function thereof.

As best seen in FIGS. 2 and 3, the cam lock 3 desirably consists of two main parts, a cylindrically shaped plug 6 and a collar or ring 7. One end 8 of the plug 6 is of a diameter substantially equal to the inner diameter of the inner telescoping member 1 for insertion therein (see FIG. 3), where it is locked in place as by indenting the inner telescoping member 1 around the outer periphery thereof. The extent to which the plug 6 may be inserted into the inner telescoping member 1 may be limited by an enlarged cylindrical portion 10 on the outer projecting portion of the plug which provides a shoulder 11 for engagement by the end of the inner member 1. Preferably, the outer diameter of the enlarged cylindrical plug portion 10 is the same as the outer diameter of the inner telescoping member 1 thus to permit a close sliding fit of the outer telescoping member 2 over such enlarged cylindrical plug and inner telescoping member 1.

Formed in the outer periphery of the enlarged cylindrical plug portion 10 is an eccentric groove 12 adapted to receive the collar 7 which has an outer cylindrical surface and an eccentric opening therein providing a collar having a shape corresponding to the shape of the eccentric groove 12, for a purpose to be subsequently described. Projecting into the eccentric groove 12 from one side thereof is a projection or stop 15 which is received in a circumferentially extending slot 18 formed in one end of the collar 7, whereby when the collar 7 and plug 6 are properly assembled, the extent of rotation of the collar 7 relative to the plug 6 is limited by engagement of the ends of the slot 16 with the stop. Alternatively, the circumferentially extending slot 16′ may be intermediate the ends of the collar 7′ and the projection or stop 15′ may be a pin projecting into the slot from the bottom of the groove 12′ as shown in FIG. 6. In any event the slot 16 desirably extends approximately 180.degree. around the collar 7, and the projection 15 is located in such a position with respect to the slot 16 that when the collar 7 is rotated in a counterclockwise direction in the groove to bring one end 17 of the slot 16 into engagement with the projection 15 as shown in FIG. 5, the collar 7 is concentrically located on the plug 16 and will not interfere with longitudinal adjustment of the outer telescoping member 2 with respect to the inner telescoping member 1. However, when the collar 7 is rotated in the reverse or clockwise direction to bring the other end 18 of the slot 18 into engagement with the projection 15 as shown in FIG. 4, the collar is eccentrically disposed on the plug, thus causing the collar and plug to be forced into frictional engagement with opposite sides of the outer telescoping member 1 for locking both telescoping members in longitudinal adjusted position. A longitudinal bore 19 through the plug 6 permits free circulation of air between the telescoping members 1 and 2 so that air pressure does not build up within the outer member 2 during retraction of the outer member and a vacuum is not created therein during extension of the outer member relative to the inner member.

The collar 7 is made of a flexible material which may be of plastic such as nylon, and is split as shown to facilitate assembly of the collar onto the plug, and insure frictional sliding contact between the collar and inner diameter of the outer member 2 even when the collar is concentric on the plug. Accordingly, rotation of the outer member 2 relative to the inner member 1 will cause the collar 7 to rotate with the outer member unless there is engagement between one end of the notch in the collar and the projection 15 on the plug precluding further rotation of the collar in an unlocking direction or the collar is already in the eccentric locking position precluding further rotation of both the collar 7 and outer member in the locking direction. The forward end 20 of the plug 6 may be tapered as shown to provide a ramp for sliding of the collar up onto the enlarged cylindrical portion 10 and into the groove 12 during assembly.

To assist in guiding of the outer member 2 along the inner member 1 during longitudinal adjustment, an end cap 21 is provided on the telescoping end of the outer member which has a close sliding fit with the outer diameter of the inner member 1 as shown in FIG. 3. A suitable legend may also be provided on the end cap 21 or adjacent thereto indicating to the operator the directions of rotation of the outer member 2 relative to the inner member 1 which result in tightening and untightening of the telescopic members.

From the foregoing, it will now be apparent that the cam lock of the present invention is easily operated by relatively rotating the telescopic members in one direction or the other to obtain locking and unlocking of such members. The lock has unidirectional characteristics providing to the user known directions for always tightening and untightening the lock, thereby substantially eliminating the chances of inadvertent overtightening or loosening of the lock.

Plastic stretch films are well known in the art (U.S. Pat. Nos. 8,435,622; 8,053,055; 7,682,683) and may take any convenient form. They may be opaque or substantially transparent. Alternatively, they may be colored, such as, for example, skin toned. The films may be composed of any suitable material, such as, for example, a polyolefin (i.e., polyethylene, polypropylene or polybutylenej, polyvinyl chloride or ethylene vinyl acetate.

Although the preferred embodiments of the invention have been illustrated and described in detail, it will be readily apparent to those skilled in the art that various modifications may be made therein without departing from the spirit of the invention or from the scope of the appended claims, For example, the system may be designed for single usage applications or for situations where multiple lengths of film are separately dispensed for applications to multiple sites; either on the same or different patients. Moreover, the film may assume any convenient shape, design or construction. For example, the film may be perforated, it being understood that the system is designed for multiple wrappings of the perforated sheet around the designated site so that the perforations overlap with non-perforated sections to provide a waterproof and protective covering. Also, those skilled in the art will appreciate that any convenient telescoping mechanism may be employed in the above described optional embodiment of the invention wherein the supply of film may be extended along the length of the handle to enable application of the film to hard-to-reach areas of the human or non-human animal undergoing treatment.

The mixture of manuka honey, activated charcoal and tea tree oil is preferably compounded in powdered form. Each of manuka honey, activated charcoal, and tea tree oil are available commercially in powdered form. Powdered manuka honey is available from Manuka Health Co. [Newmarket, Aukland, New Zealand]. Powdered tea tree oil and powdered activated charcoal may be obtained from Alibaba Group [San Mateo, Calif.].

The mixture, in its broadest aspect comprises, by weight, from about 5 to about 25% of powdered manuka honey, from about 15 to about 35% of powdered tea tree oil, and the remainder, powdered activated charcoal.

Preferably, the mixture comprises, by weight, from about 10 to about 20% of powdered manuka honey, from about 20 to about 30% of powdered tea tree oil, and the remainder, powdered activated charcoal.

Most preferably, the mixture comprises, by weight, from about 12 to about 18% of powdered manuka honey, from about 23 to about 27% of powdered tea tree oil, and the remainder, powdered activated charcoal.

The mixture may be applied to the film according to any convenient method; i.e., powder coating, solvent coating, and the like. Preferably, the mixture is admixed with a suitable physiologically acceptable carrier liquid, such as, for example, coconut oil, and applied to the surface of the film and allowed to dry. Typically, an emulsifying agent such as glycerin, mustard, or the like is employed to obtain a homogenous mixture suitable for coating the film. The thus coated film would be allowed to dry, with or without the addition of a drying accelerant, or a the wet coating may be overcoated with gelatin or other physiologically acceptable solid material which would allow the coated film to be wound onto the spool of the above-described application systems, and which would melt or dissolve after application to the human or non-human animal.

Those skilled in the art will appreciate that the mixture may be also be directly applied to a surface of a human or non-human animal to achieve wound healing and moisture-proofing.

The thickness of the coating on the film comprising the mixture of manuka honey, activated charcoal, and tea tree oil may range from about 0.01 millimeters to about 5 millimeters, more preferably from about 0.05 millimeters to about 2 millimeters.

When applied to the human or non-human animal in need thereof, the moisture of the surface of and/or the body heat of the human or non-human animal to which the mixture is applied activates the wound healing and moisture-proofing properties thereof.

It will be understood by those skilled in the art that the mixture of the invention may also contain other ingredients which are known to enhance the skin of the human or non-human animal to which it is applied, including, but not limited to:

Lidocaine HCl: a topical pain reliever; Chloroxylenol: an antiseptic; Petrolatum: A topical skin protecting agent; Jojoba Seed Oil: Natural moisturizer and healing agent; Beeswax: Protects the skin from irritants, draws moisture to the skin and helps seal it in; Macadamia Seed Oil: Emollient and skin protector; Soybean Oil: Natural moisturizer used to hydrate the skin and help the skin retain moisture; Lavender Oil: Antiseptic and antifungal agent; Cannabis Seed Oil: Contains certain lipids the promote healthy skin; Aloe Leaf Extract: Hydrates, protects the skin; Sunflower Seed Oil: Rich in beta-carotene which improves the appearance of the skin, and minimizes scars; Vitamin A: promotes healthy skin; Vitamin C: Aids in healing and rejuvenation of the skin; Vitamin E: Anti-oxidant that helps prevent free radical damage caused by the sun and other environmental effects; Squalane: Promotes youthful skin, and the healing of scar tissue.

Although the preferred embodiments of the invention have been illustrated and described in detail, it will be readily apparent to those skilled in the art that various modifications may be made therein without departing from the spirit of the invention or from the scope of the appended claims.

1. A system for providing a protective covering for at least a portion of a human or non-human animal comprising a supply of dispensable plastic stretch film having a predetermined thickness and width and a dispenser-applicator for dispensing a predetermined length of film from the supply thereof and applying the predetermined length of film to the human or non-human animal wherein the dispenser-applicator is, optionally, coupled to and longitudinally aligned with a telescoping mechanism which enables extension of the supply of dispensable plastic stretch film, and wherein the surface of the side of film applied to the human or non-human animal is at least partially coated with a mixture of manuka honey, activated charcoal, and tea tree oil.

2. The system of claim 1 wherein said supply of plastic stretch film comprises a roll of said film.

3. The system of claim 1 wherein said plastic stretch film is substantially transparent.

4. The system of claim 1 wherein said plastic stretch film is colored.

5. The system of claim 4 wherein said plastic stretch film is a skin tone color.

6. The system of claim 1 wherein said plastic stretch film comprises a polyolefin, polyvinyl chloride or ethylene vinyl acetate.

7. The system of claim 6 wherein said polyolefin comprises polyethylene, polypropylene or polybutylene.

8. An article of manufacture comprising packaging material and the system of claim 1 contained within said packaging material, and wherein said packaging material comprises a label which indicates that said system can be used for providing a protective covering for at least a portion of a human or non-human animal.

9. The article of manufacture of claim 8 wherein said packaging material provides protection for said contained system from contamination by water and/or microorganisms.

10. A wound-healing composition of matter comprising a mixture of powdered manuka honey, powdered activated charcoal, and powdered tea tree oil.

11. The composition of claim 10 in combination with a physiologically acceptable carrier.

12. The composition of claim 11, wherein said carrier is a liquid.

13. The composition of claim 12, wherein said liquid carrier is an oil.

14. The composition of claim 13, wherein said oil is a vegetable oil.

15. The composition of claim 13, wherein said vegetable oil is coconut oil.

16. The composition of claim 10 comprising, by weight, from about 5 to about 25% of powdered manuka honey, from about 15 to about 35% of powdered tea tree oil, and the remainder, powdered activated charcoal.

17. The composition of claim 10 comprising, by weight, from about 10 to about 20% of powdered manuka honey, from about 20 to about 30% of powdered tea tree oil, and the remainder, powdered activated charcoal.

18. The composition of claim 10 comprising, by weight, from about 12 to about 18% of powdered manuka honey, from about 23 to about 27% of powdered tea tree oil, and the remainder, powdered activated charcoal.

19. A method for providing a protective covering for at least a portion of a human or non-human animal comprising dispensing a predetermined length of film from the system of claim 1 and applying it to said portion of said human or non-human animal.

20. A method for treating a wound on a human or non-human animal comprising applying to said wound a wound-healing amount of the composition of claim 10
